(19) United States
(12) Patent Application Publication
(10) Pub. No.: US 2018/0104013 A1
(43) Pub. Date: Apr. 19, 2018

(54) ACTUATOR

(71) Applicants: Sharp Kabushiki Kaisha, Sakai City, Osaka (JP); Public University Corporation Hiroshima City University, Hiroshima-shi, Hiroshima (JP)

(72) Inventors: Masaki HAMAMOTO, Sakai City (JP); Hideki ETOH, Sakai City (JP); Kazuhiro TANIGUCHI, Hiroshima-shi (JP); Masazumi OKAJIMA, Hiroshima-shi (JP)

(21) Appl. No.: 15/573,518

(22) PCT Filed: May 10, 2016
(86) PCT No.: PCT/JP2016/063874

§ 371 Date: Nov. 13, 2017

(30) Foreign Application Priority Data
May 21, 2015 (JP) 2015-103889

(51) Int. Cl. A61B 34/00; A61B 34/37; A61B 1/005

(52) U.S. Cl. CPC A61B 34/77; A61B 34/37; A61B 90/361; A61B 1/0052; A61B 2034/742; A61B 34/71

(57) ABSTRACT

Provided is an actuator that is able to adjust a ratio of a speed in a rectilinear-direction and a speed in a rotation-direction of an operation element. A differential drive mechanism (110A) includes a front conveyance roller (1112) and a rear conveyance roller (1113) that are able to convey a rod-shaped insertion unit in a long axis direction thereof and rotate the insertion unit about the long axis, and an angle changing mechanism that changes crossing angles of the front conveyance roller and the rear conveyance roller with respect to the insertion unit.

### TECHNICAL FIELD

The present invention relates to an actuator that operates a rod-shaped operation element.

### BACKGROUND ART

For labor-saving in a surgical procedure, a medical robot has been increasingly introduced. A field of the medical robot is classified into a large-sized, multi-degree-of-freedom, and multi-functional one whose main purpose is the substitution for a human arm and which is obtained by applying an arm robot for industrial use, and a single-function one specialized in a simple purpose such as catheter delivery.

As an example of the latter, NPL 1 discloses an actuator (vessel catheter insertion module) that delivers or rotates a catheter by using a friction wheel mechanism. The actuator has a mechanism by which delivery or rotation of an operation element such as a catheter is controlled in accordance with rotation directions of two rollers arranged obliquely to a cylinder.

### CITATION LIST

### Non Patent Literature

NPL 1: “Catheter Insertion Module Using Friction Wheel Mechanism for Vascular Surgery” in Journal of the Japan Society of Computer Aided Surgery, Vol. 15 (2013), No. 2, Special Number: 22nd Annual Congress of Japan Society of Computer Aided Surgery, p. 162-163

### SUMMARY OF INVENTION

### Technical Problem

However, since it is difficult for the actuator of NPL 1 to adjust a ratio between a speed in a rectilinear-direction and a speed in a rotation-direction of the operation element, there is a problem in practical use. This will be described below specifically.

In NPL 1, as illustrated in drawings, rollers each having a diameter significantly greater with respect to a diameter of the catheter are used and a crossing angle between a rotation axis of each of the rollers and the direction of catheter delivery is as small as π/6. In the configuration, the ratio of the rotation speed to the delivery is thus significantly great. Such a configuration is convenient for a catheter that is inserted while being kinked.

However, there arises a problem that suitability is different in an endoscope or the like. For example, in an endoscope, such as an oblique-viewing endoscope, having a configuration in which a camera is attached horizontally relative to an insertion direction, while a suitable speed in a delivery direction is substantially several cm to 1 m/second, a suitable speed in a rotation direction is substantially 90°/second at most. In a case where an endoscope whose outer diameter is about 5 mm, which is general as of this writing, is conveyed, according to calculation by inventors of the invention, when the crossing angle is π/6 as described above, the speed in the rotation direction is as significantly high as about 8 rotations per second while a speed in a translation direction is 10 cm/second. Thus, it becomes very difficult to perform an operation of placing a surgical part at the center of a visual field.

That is, an actuator in past instances has a suitable configuration for a catheter that is inserted while being kinked, but has a problem that use for an operation of an endoscope is difficult because a rotation speed is too high.

There is also a problem that operation elements having different outer diameters have different rotation speeds.

Thus, the actuator of NPL 1 has a problem that since a wheel speed at which the rollers are wheeled needs to be changed every time in accordance with a wheel direction of an operation element on the basis of suitability for usage and an outer diameter of the operation element, a control system is complicated.

The invention was made in view of the aforementioned problems, and an object thereof is to provide an actuator that is able to easily adjust a ratio between a speed in a rectilinear-direction and a speed in a rotation-direction of an operation element.

### Solution to Problem

In order to solve the aforementioned problems, an actuator according to an aspect of the invention is an actuator including a plurality of conveyance rollers that are able to convey a rod-shaped operation element in a long axis direction thereof and rotate the operation element about the long axis, and the actuator includes an angle changing mechanism that changes crossing angles of the plurality of conveyance rollers with respect to the operation element.

### Advantageous Effects of Invention

According to an aspect of the invention, it is possible to easily adjust a ratio of a speed in a rectilinear-direction and a speed in a rotation-direction of an operation element.

### BRIEF DESCRIPTION OF DRAWINGS

FIG. 1 is a schematic view illustrating a use form of a medical device according to an embodiment of the invention.

FIG. 2 is a perspective view illustrating an external appearance of an insertion unit conveyance unit according to the embodiment of the invention.

FIG. 3 is a schematic view illustrating a schematic configuration of the insertion unit conveyance unit according to the embodiment of the invention.

FIG. 4 is a schematic view illustrating a form of an operation of the insertion unit conveyance unit according to the embodiment of the invention.

FIG. 5 is a schematic view illustrating another form of the operation of the insertion unit conveyance unit according to the embodiment of the invention.

FIG. 6 illustrates positions of conveyance rollers on guides of a differential drive mechanism used in the medical device according to the embodiment of the invention.

FIGS. 7(a) and (b) both illustrate a resultant vector of frictional force by conveyance rollers.

FIG. 8 is a schematic view illustrating a schematic configuration of an in-wheel motor used for the insertion unit conveyance unit according to the embodiment of the invention.

FIG. 9 is a schematic view illustrating a schematic configuration of an ultrasonic vibrator used for the in-wheel motor used for the insertion unit conveyance unit according to the embodiment of the invention.

FIG. 10 is a schematic view illustrating a vibration mode of the ultrasonic vibrator according to the embodiment of the invention.

FIG. 11 is a schematic view illustrating another vibration mode of the ultrasonic vibrator according to the embodiment of the invention.

FIGS. 12(a) and (b) both illustrate a conveyance principle of a rotor of the ultrasonic vibrator according to the embodiment of the invention.

FIG. 13 is a schematic view illustrating an overview of a preloading holding mechanism by which the ultrasonic vibrator according to the embodiment of the invention is pressed against the rotor, in which (a) illustrates a state before an adjustment screw is tightened and (b) illustrates a state after the adjustment screw is tightened.

FIG. 14 is a schematic view illustrating a schematic configuration of an insertion unit conveyance unit used for a medical device according to a second embodiment of the invention.

FIGS. 15 (a) and (b) both illustrate a resultant vector of frictional force by conveyance rollers.

FIG. 16 is a schematic view illustrating an operation principle of a function of an insertion unit conveyance unit used for a medical device according to a third embodiment of the invention.

FIG. 17 is a schematic view illustrating a schematic configuration of an insertion unit conveyance unit used for a medical device according to a fourth embodiment of the invention.

### DESCRIPTION OF EMBODIMENTS

### Embodiment 1

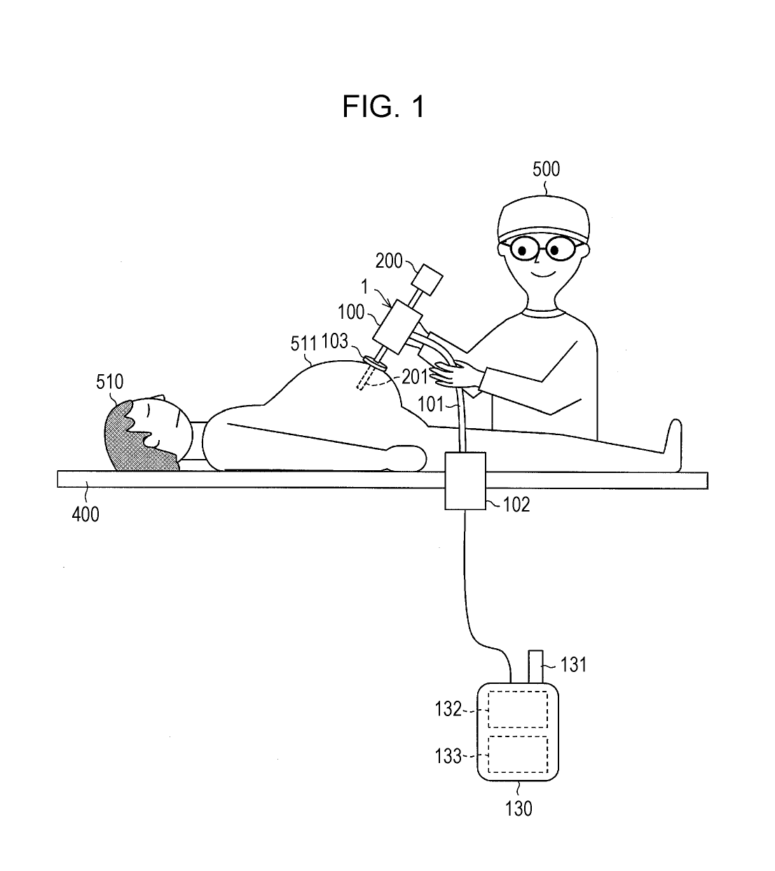

An embodiment of the invention will be described in detail below with reference to FIGS. 1 to 13.

### (Outline of Medical Device 1)

FIG. 1 is a schematic view illustrating an example of a use form of a medical device 1 according to the embodiment of the invention. The medical device 1 is a device that adjusts a position of a rigid endoscope 200. In the present embodiment, as an example to which the invention is applied, it is assumed that an insertion unit (sheath tube) 201 of the rigid endoscope 200 is inserted into a body cavity of an abdomen 511 of a patient 510 lying on an operating table 400 so that a surgeon 500 performs treatment on the basis of an image obtained from an image sensor positioned at a distal end of the insertion unit 201.

In FIG. 1, the medical device 1 includes an insertion unit conveyance unit 100, a flexible arm (actuator fixing unit) 101, a stand (actuator fixing unit) 102, a surgical port 103, a controller unit (control device) 130, and the rigid endoscope 200. Note that, the insertion unit conveyance unit 100 and the controller unit 130 will be described in detail later.

The flexible arm 101 has one end supporting and fixing the insertion unit conveyance unit 100 and is able to be bent by a hand so as to have a desired shape. That is, the flexible arm 101 is used to arrange and fix the insertion unit conveyance unit 100 at a position desired by the surgeon 500.

The stand 102 fixes the other end of the flexible arm 101 to thereby fix the flexible arm 101 to a side of the patient 510 lying on the operating table 400. The stand 102 is installed in (fixed to) the operating table 400.

The surgical port 103 is a medical instrument having a through-hole through which a medical instrument is inserted into a body cavity of the patient 510, and is arranged on a surface of the abdomen 511 of the patient 510. Note that, the surgical port 103 does not need to be always used depending on an operative procedure, and is not an indispensable component in the present embodiment.

In the present embodiment, the rigid endoscope 200 having the cylindrical (rod-shaped) insertion unit 201 is used as an example of the medical instrument, but the medical instrument is not limited thereto. Instead of the rigid endoscope 200, it is possible to use a medical instrument having an rod-shaped (columnar) insertion unit for inserting a medical instrument into a body of the patient 510. For example, one having a surgical instrument such as a forceps disposed in a distal end of the columnar insertion unit, a columnar catheter also serving as the insertion unit, or the like is able to be used as the medical instrument. These are generally referred to as an operation element in the invention.

### (Configuration of Insertion Unit Conveyance Unit 100)

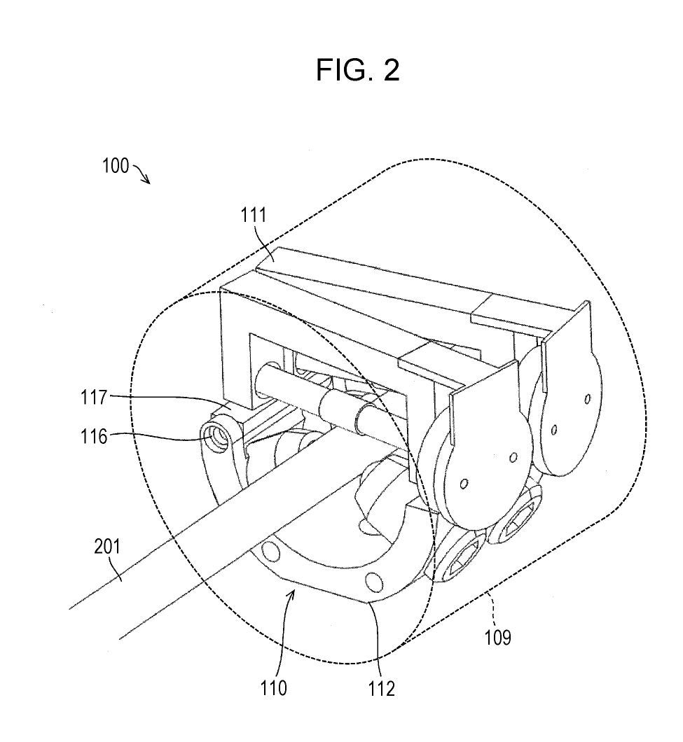

FIG. 2 is a perspective view illustrating a schematic configuration of the insertion unit conveyance unit 100. As illustrated in FIG. 2, the insertion unit conveyance unit 100 includes an actuator holding unit (actuator fixing unit) 109 and a differential drive mechanism (actuator, friction drive actuator) 110. The differential drive mechanism 110 includes a plurality of conveyance rollers (a front conveyance roller 1112 and a rear conveyance roller 1113) that are able to convey the insertion unit (operation element) 201 in the rod shape in a long axis direction thereof and rotate the insertion unit 201 about the long axis.

The actuator holding unit 109 is a hollow housing which holds the differential drive mechanism 110, and an end of the flexible arm 101 (refer to FIG. 1) is fixed to a side surface of the actuator holding unit 109. The actuator holding unit 109, the flexible arm 101, and the stand 102 form the actuator fixing unit for fixing the differential drive mechanism 110 to a vicinity of a surgical site.

Note that, to make the drawing easy to be understood, a spring 111a and guides (restriction units) 1130 and 1131 described below are omitted in FIG. 2.

### (Configuration of Differential Drive Mechanism 110A)

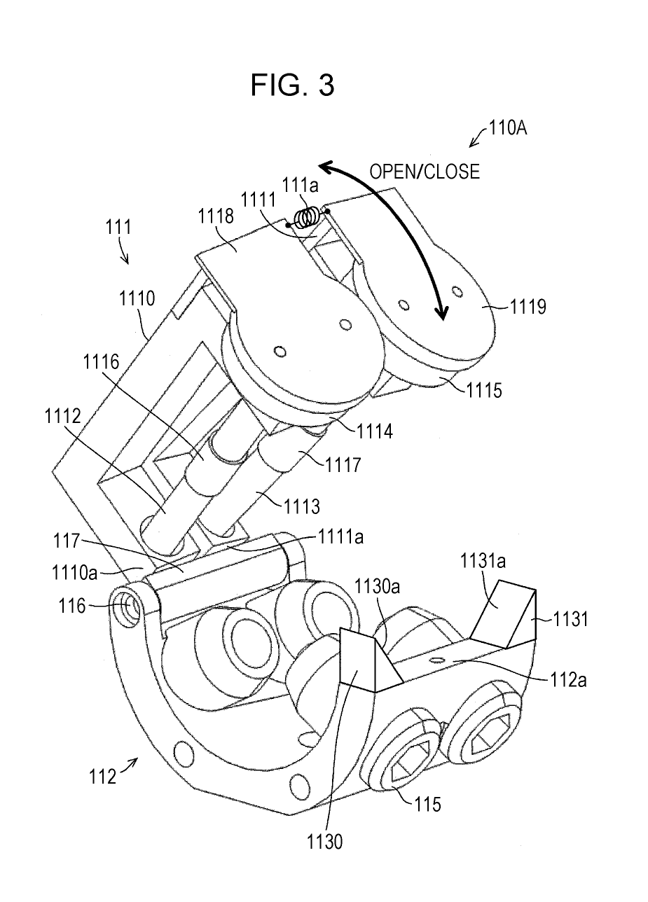

FIG. 3 is a perspective view illustrating a schematic configuration of a differential drive mechanism 110A as an example of the differential drive mechanism 110 illustrated in FIG. 2. As illustrated in FIG. 3, the differential drive mechanism 110A includes an upper housing unit (first unit) 111, a lower housing unit (second unit) 112, a coupling unit 117, and a preloading spring (restoring unit) 116.

The upper housing unit 111 includes a front arm 1110, a rear arm 1111, and the spring (urging unit) 111a. The front arm 1110 and the rear arm 1111 are configured so as to be rotatable with connection units 1110a and 1111a to the lower housing unit 112 as axes.

The front arm 1110 includes the front conveyance roller 1112, a front in-wheel motor (ultrasonic actuator, ultrasonic motor) 1114 that drives the front conveyance roller 1112, and a rubber roller 1116. The rear arm 1111 includes the rear conveyance roller 1113, a rear in-wheel motor (ultrasonic actuator, ultrasonic motor) 1115 that drives the rear conveyance roller 1113, and a rubber roller 1117.

The rubber rollers 1116 and 1117 are elastic friction members that are arranged on surfaces of the front conveyance roller 1112 and the rear conveyance roller 1113, respectively. Thus, frictional force between the insertion unit 201 and each of the conveyance rollers increases, so that idling of the conveyance rollers is able to be prevented.

The rubber rollers 1116 and 1117 are arranged so as to be detachable from the front conveyance roller 1112 and the rear conveyance roller 1113, respectively. Thus, the robber rollers 1116 and 1117 are able to be easily replaced in a case of damage or contamination. Therefore, a friction member for which sterilization at high temperature is not able to be performed is also able to be used.

Specifically, each of the rubber rollers 1116 and 1117 may have a groove substantially parallel to the long axis of the insertion unit 201. It may be configured so that the rubber rollers 1116 and 1117 are able to be pulled out while the front in-wheel motor 1114 and the rear in-wheel motor 1115 are being detached from the front conveyance roller 1112 and the rear conveyance roller 1113.

On surfaces of the front conveyance roller 1112 and the rear conveyance roller 1113, recessed steps whose widths are respectively equal to or greater than widths of the rubber rollers 1116 and 1117 are provided. Therefore, without using adhesive or the like, positions of the rubber rollers 1116 and 1117 are able to be prevented from being significantly shifted due to friction with the insertion unit 201.

The spring 111a is a spring whose both ends are connected to the front arm 1110 and the rear arm 1111 and is configured so as to keep the front arm 1110 and the rear arm 1111 away from each other.

The lower housing unit 112 includes four ball bearings (holding units, sliding bodies) 115 and the guides (restriction units) 1130 and 1131.

The ball bearings 115 hold the insertion unit 201 between the front conveyance roller 1112 and the rear conveyance roller 1113.

The guides 1130 and 1131 are a pair of members for guiding the front in-wheel motor 1114 and the rear in-wheel motor 1115 in a process that the upper housing unit 111 and the lower housing unit 112 are closed. The guides 1130 and 1131 are arranged on an end surface 112a of the lower housing unit 112, against which the front in-wheel motor 1114 and the rear in-wheel motor 1115 abut in a state where the upper housing unit 111 and the lower housing unit 112 are closed.

The guides 1130 and 1131 respectively have inclined surfaces 1130a and 1131a that contact the front in-wheel motor 1114 and the rear in-wheel motor 1115 in the process that the upper housing unit 111 and the lower housing unit 112 are closed. The inclined surfaces 1130a and 1131a are placed at positions facing each other and a distance between the inclined surface 1130a and the inclined surface 1131a decreases as being closer to the end surface 112a.

Thus, as a distance between the upper housing unit 111 and the lower housing unit 112 is shorter, the front in-wheel motor 1114 and the rear in-wheel motor 1115 are guided by the inclined surfaces 1130a and 1131a, so that a distance between the front in-wheel motor 1114 and the rear in-wheel motor 1115 decreases, resulting in reduction in an angle formed by the front conveyance roller 1112 and the rear conveyance roller 1113.

The coupling unit 117 is a member that has a function as a hinge coupling the first and second units so that relative positions of the upper housing unit 111 and the lower housing unit 112 are able to be changed. The distance between the upper housing unit 111 and the lower housing unit 112 that are coupled by the coupling unit 117 varies in accordance with a thickness of the insertion unit 201. That is, the coupling unit 117 varies distances between the respective front conveyance roller 1112 and rear conveyance roller 1113 and the ball bearings 115 in accordance with the thickness of the insertion unit 201.

The preloading spring 116 applies restoring force in a direction in which the upper housing unit 111 and the lower housing unit 112 are closed together. When being closed together, the upper housing unit 111 and the lower housing unit 112 constitute an annular housing.

When the upper housing unit 111 and the lower housing unit 112 are closed together, the restoring force of the preloading spring 116 presses the rubber roller 1116 in the front conveyance roller 1112, the rubber roller 1117 in the rear conveyance roller 1113, and the four ball bearings 115 against a side surface of the insertion unit 201. Hereinafter, unless otherwise noted, the rollers also including the rubber roller units are referred to as conveyance rollers for simplification of description.

The conveyance rollers are arranged in the respective arms so as to be rotatable via bearings that are not illustrated. The upper housing unit 111 and the lower housing unit 112 are coupled so as to be openable and closable. The differential drive mechanism 110 conveys the insertion unit 201 of the rigid endoscope 200 in a translation or rotation direction in a state where the actuator holding unit 109 fixes a position of the differential drive mechanism 110 with respect to a surgical site in a body cavity. The translation direction is a direction parallel to the long axis direction of the insertion unit 201 and the rotation direction is a rotation direction about the long axis of the insertion unit 201. Note that, the rigid endoscope 200 is constituted by a grip unit and the insertion unit 201 and the insertion unit 201 has a cylindrical shape.

According to the aforementioned configuration, the insertion unit 201 of the rigid endoscope 200 has a direction vertical to the axis direction of the insertion unit 201 restricted by the front conveyance roller 1112, the rear conveyance roller 1113, and two ball bearings 115 (FIG. 2).

On the other hand, when the upper housing unit 111 and the lower housing unit 112 are opened together, the front conveyance roller 1112 and the rear conveyance roller 1113 are separated from the ball bearings 115, so that the insertion unit 201 is released from the differential drive mechanism 110. In this manner, it is possible to remove the rigid endoscope 200 from the insertion unit conveyance unit 100 to perform cleaning or the like for the rigid endoscope 200 alone.

Here, the ball bearings 115 come into point contact with the side surface of the insertion unit 201. Therefore, though usage of the two ball bearings 115 in addition to the front conveyance roller 1112 and the rear conveyance roller 1113 is satisfactory, the four ball bearings are used here in consideration of position adjustment of an operation of the insertion unit 201. In addition, much more ball bearings may be used.

### (Driving Principle of Insertion Unit 201)

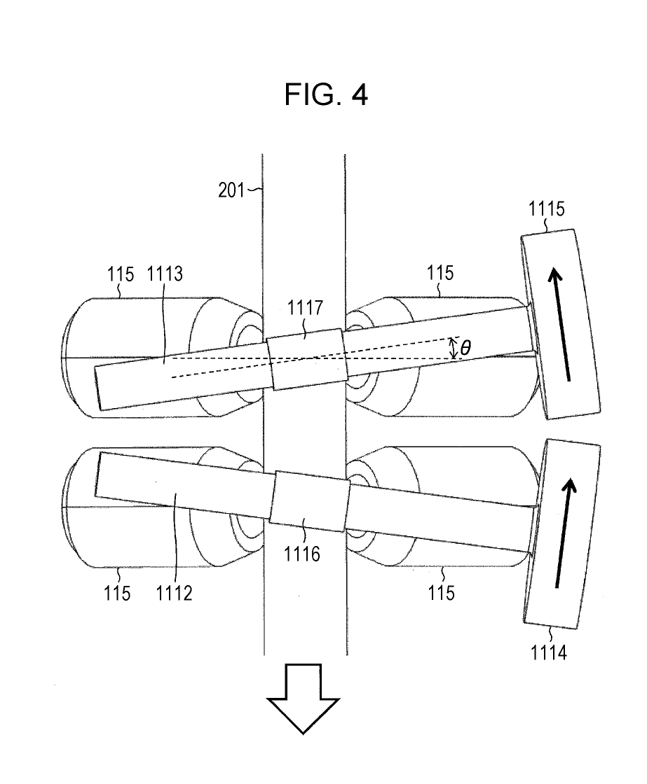

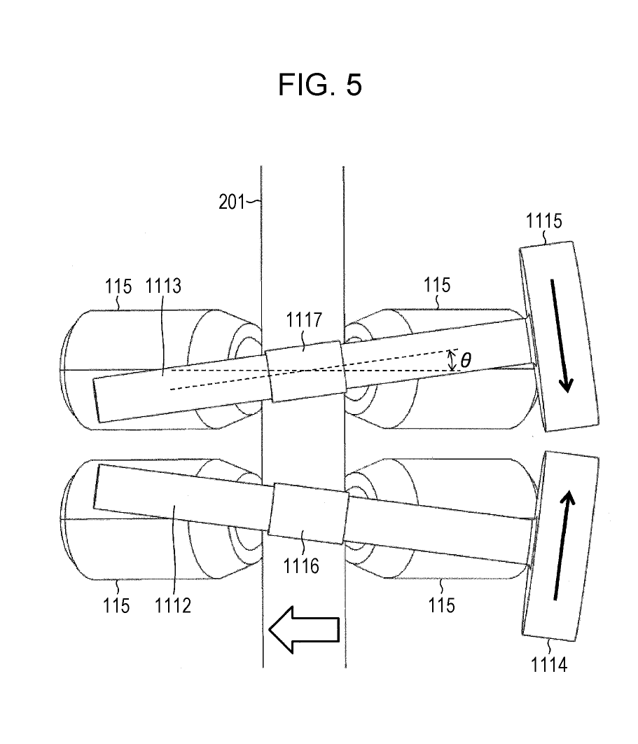

FIGS. 4 and 5 each illustrates a form of an operation of the insertion unit conveyance unit 100. In the present embodiment, translation and rotation operations of the insertion unit 201 are realized by differential drive similarly to the invention of PTL 1. That is, when both the conveyance rollers rotate in the same direction as illustrated in FIG. 4, the insertion unit 201 is conveyed in the translation direction with resultant force of frictional force applied to the insertion unit 201. When both the conveyance rollers rotate in reverse directions as illustrated in FIG. 5, the insertion unit 201 is conveyed in the rotation direction with the resultant force of the frictional force applied to the insertion unit 201.

At this time, when a diameter of the conveyance rollers is ϕ, a crossing angle is θ, a rotation speed of the conveyance rollers is ω, and a diameter of the insertion unit 201 is D,

the delivery speed in the case of rotation in the same direction is provided by

vₜᵣₐₙₛ=π*ϕ*ω*cos(θ), and

the rotation speed in the case of the rotation in the reverse directions is provided by

vᵣₒₜ=ϕ*ω*sin(θ)/D.

The crossing angle θ is an angle between the rotation axis of each of the conveyance rollers and a normal line of the long axis of the insertion unit 201.

The differential drive mechanism 110A according to the present embodiment includes an angle changing mechanism that changes the crossing angles of the front conveyance roller 1112 and the rear conveyance roller 1113 with respect to the insertion unit 201.

The angle changing mechanism according to the present embodiment includes the spring 111a that urges the front conveyance roller 1112 and the rear conveyance roller 1113 so that a distance between one distal end of the front conveyance roller 1112 and one distal end of the rear conveyance roller 1113 increases, and the guides 1130 and 1131 that restrict the distance between the distal ends in accordance with the distances from the respective front conveyance roller 1112 and rear conveyance roller 1113 to the ball bearings 115.

The angle changing mechanism changes crossing angles θ in conjunction with the distances from the respective front conveyance roller 1112 and rear conveyance roller 1113 to the ball bearings 115, which are changed by the coupling unit 117. The angle changing mechanism changes the crossing angles so that the crossing angle θ by the front conveyance roller 1112 and the crossing angle θ by the rear conveyance roller 1113 are the same with each other.

### (Operation of Angle Changing Mechanism)

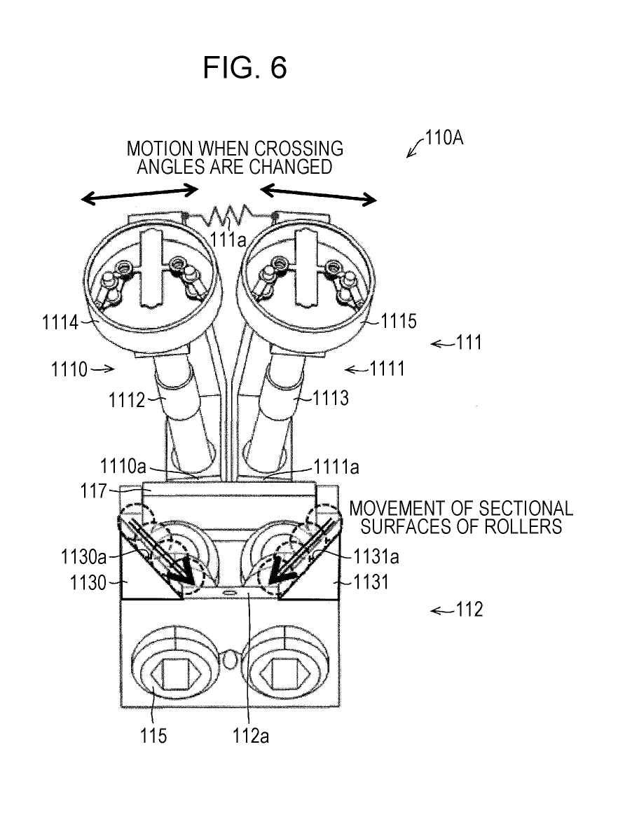

FIG. 6 illustrates positions of the front conveyance roller 1112 and the rear conveyance roller 1113 on the inclined surfaces 1130a and 1131a. Though FIG. 6 illustrates a configuration in which the front conveyance roller 1112 and the rear conveyance roller 1113 contact the inclined surfaces 1130a and 1131a, it may be configured so that the front in-wheel motor 1114 and the rear in-wheel motor 1115 contact the inclined surfaces 1130a and 1131a.

In the present embodiment, the inclined surfaces 1130a and 1131a are arranged at symmetrical positions with a plane vertical to the long axis of the insertion unit 201 as a reference plane. Thus, the front in-wheel motor 1114 and the rear in-wheel motor 1115 (or ends on in-wheel motor sides of the front conveyance roller 1112 and the rear conveyance roller 1113) are guided to be symmetrical with respect to the reference plane.

When an outer diameter of the insertion unit 201 is large, the upper housing unit 111 and the lower housing unit 112 are separated from each other. Thus, the front in-wheel motor 1114 and the rear in-wheel motor 1115 move away from the end surface 112a, so that the front in-wheel motor 1114 and the rear in-wheel motor 1115 (or the aforementioned ends) are at positions where an interval between the inclined surfaces 1130a and 1131a is large. At this time, due to an operation of the spring 111a, the front in-wheel motor 1114 and the rear in-wheel motor 1115 are urged in directions to be farther from each other and an angle formed by the front conveyance roller 1112 and the rear conveyance roller 1113 becomes large. As a result, the crossing angles θ between the normal line of the long axis of the insertion unit 201 and the respective front conveyance roller 1112 and rear conveyance roller 1113 become large (refer to FIG. 4).

On the other hand, when the outer diameter of the insertion unit 201 is small, the upper housing unit 111 and the lower housing unit 112 come close to each other. Thus, the front conveyance roller 1112 and the rear conveyance roller 1113 are respectively guided by the guides 1130 and 1131, so that the front arm 1110 and the rear arm 1111 come close to each other. At this time, the crossing angles θ become small.

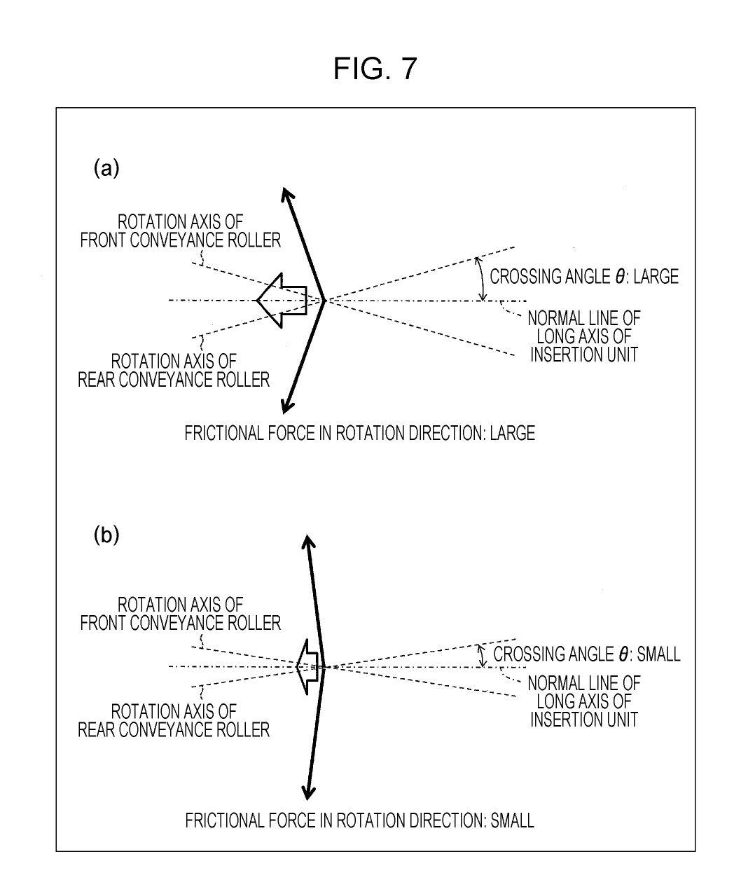

FIGS. 7(a) and (b) each illustrates a resultant vector, in a direction in which the insertion unit 201 rotates, of frictional force generated through rotation of the front conveyance roller 1112 and the rear conveyance roller 1113.

When the crossing angles θ are large, the resultant vector, in the direction in which the insertion unit 201 rotates, of the frictional force generated through rotation of the front conveyance roller 1112 and the rear conveyance roller 1113 is large as illustrated in FIG. 7(a). Thus, when the front conveyance roller 1112 and the rear conveyance roller 1113 rotate at a predetermined speed, a rotation speed of the insertion unit 201 increases.

When the outer diameter of the insertion unit 201 is large, the crossing angles θ are automatically set to be large in the differential drive mechanism 110A. Thus, when the outer diameter of the insertion unit 201 is large, the rotation speed of the insertion unit 201 increases.

On the other hand, when the crossing angles θ are small, the resultant vector, in the direction in which the insertion unit 201 rotates, of the aforementioned frictional force is small as illustrated in FIG. 7(b), so that when the front conveyance roller 1112 and the rear conveyance roller 1113 rotate at the predetermined speed, the rotation speed of the insertion unit 201 decreases.

When the outer diameter of the insertion unit 201 is small, the crossing angles θ are automatically set to be small in the differential drive mechanism 110A. Thus, when the outer diameter of the insertion unit 201 is small, the rotation speed of the insertion unit 201 decreases.

A relation of the outer diameter of the insertion unit 201 and the crossing angles θ is able to be appropriately set in accordance with an interval between the guides 1130 and 1131 and shapes thereof, in particular, inclination angles of the inclined surfaces relative to the end surface 112a.

Note that, when force by the spring 111a is larger than force by the preloading spring 116, the front arm 1110 and the rear arm 1111 may be guided by the guides 1130 and 1131 and rise from the insertion unit 201. Thus, the force by the spring 111a is preferably smaller than the force by the preloading spring 116.

### (In-Wheel Motor)

### (Entire Configuration)

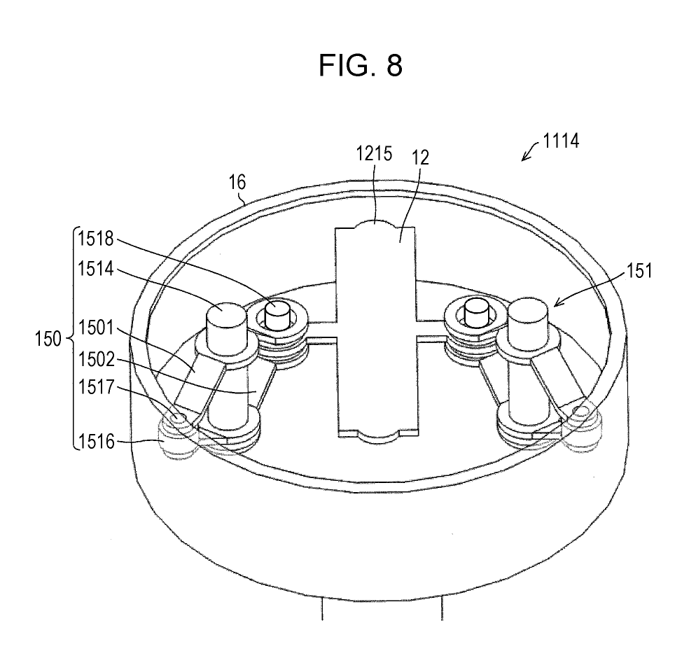

FIG. 8 illustrates a configuration of the front in-wheel motor 1114. Note that, a configuration of the rear in-wheel motor 1115 is similar to the configuration of the front in-wheel motor 1114, and is thus not described with use of the drawing.

As illustrated in FIGS. 8 and 3, the front in-wheel motor 1114 includes an ultrasonic vibrator 12, pantograph preloading mechanisms 150 and 151, a housing 16, and a motor cover 1118. The rear in-wheel motor 1115 includes a motor cover 1119 instead of the motor cover 1118.

Each of the front in-wheel motor 1114 and the read in-wheel motor 1115 is configured so that the ultrasonic vibrator 12 that has a function of conveying the housing (rotor) 16 by a distal end being elliptically moved is pressed against the housing 16 by each of two pairs of pantograph preloading mechanisms 150 and 151.

Each of the two pairs of pantograph preloading mechanisms 150 and 151 holds the ultrasonic vibrator 12 with a node of vibration thereof and generates pressure for pressing the ultrasonic vibrator 12 against the housing 16. The pantograph preloading mechanisms 150 and 151 are fixed to the motor cover 1118 (or the motor cover 1119) and the motor cover 1118 (or the motor cover 1119) is fixed to the front arm 1110 (or the rear arm 1111).

It is configured so that the housing 16 is held rotatably with respect to the front arm 1110 (or the rear arm 1111), and thus the housing rotates with respect to the front arm 1110 and the rear arm 1111 by frictional force applied from the ultrasonic vibrator 12 to the housing 16.

### (Main Configuration of Ultrasonic Vibrator 12)

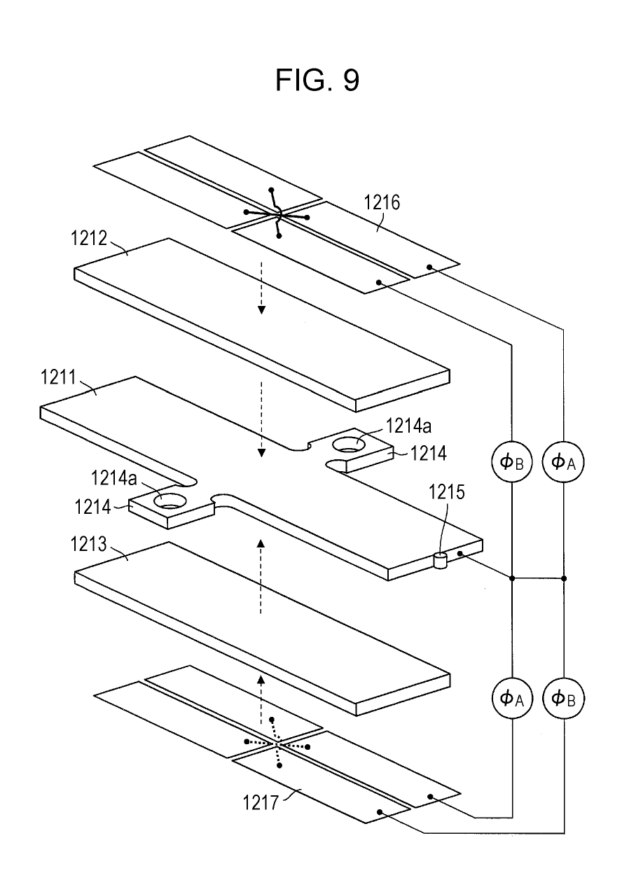

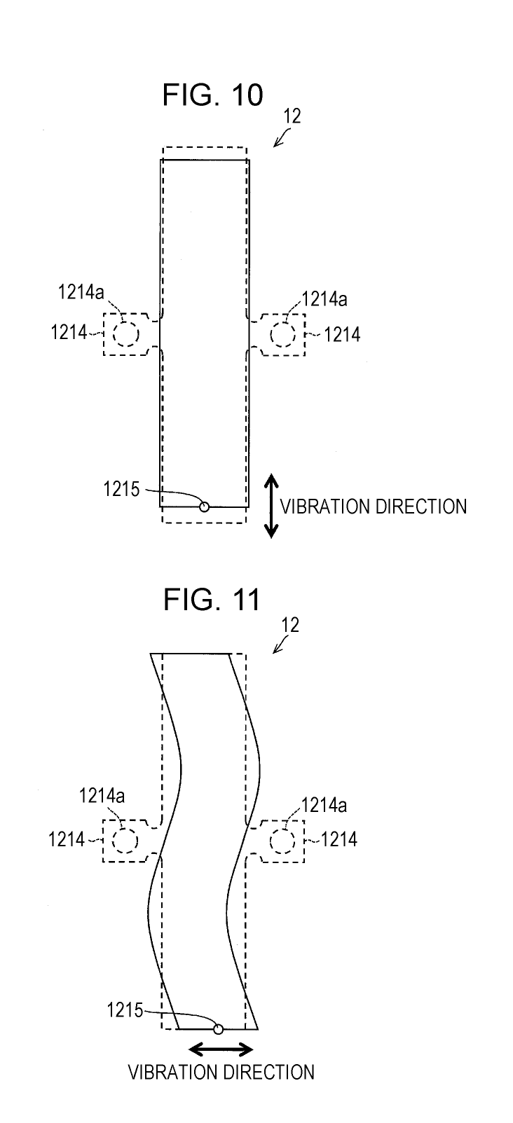

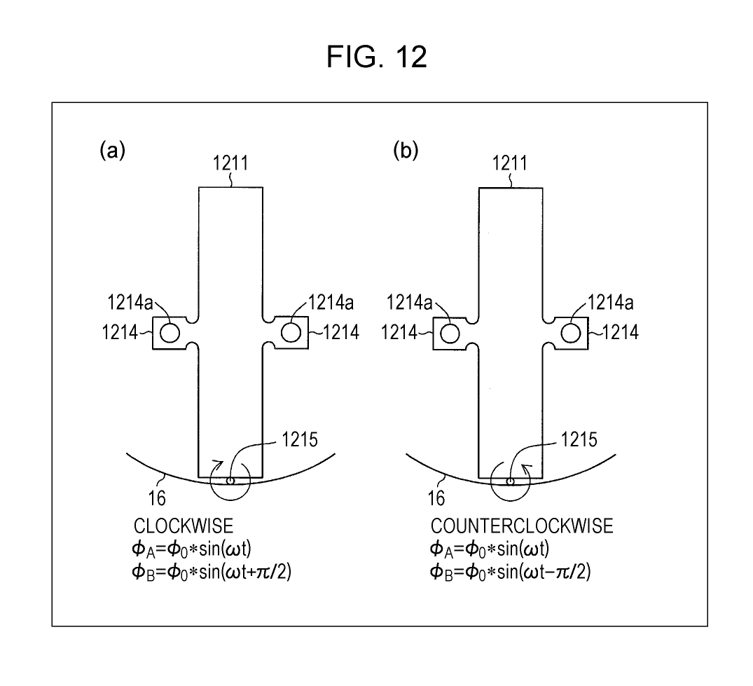

A representative configuration and function of the ultrasonic vibrator 12 used in the insertion unit conveyance unit 100 according to the present embodiment will be described with reference to FIGS. 9 to 12. FIG. 9 is a schematic view illustrating a schematic configuration of the ultrasonic vibrator 12. FIGS. 10 and 11 are schematic views each illustrating a vibration mode of the ultrasonic vibrator 12. FIG. 12 is a schematic view illustrating a principle of rotation of the housing (also serving as the rotor) 16 by the ultrasonic vibrator 12.

As illustrated in FIG. 9, the ultrasonic vibrator 12 includes a vibration plate 1211, an upper PZT (Lead Zirconate Titanate) element 1212, a lower PZT element 1213, an upper electrode 1216, and a lower electrode 1217.

The ultrasonic vibrator 12 has the upper PZT element 1212 in a rectangular shape and the lower PZT element 1213 in a rectangular shape placed on both surfaces of the vibration plate 1211 in a substantially rectangular shape. The upper PZT element 1212 has the upper electrode 1216, which is divided into four pieces, placed on a surface opposite to the vibration plate 1211 and the lower PZT element 1213 has the lower electrode 1217, which is divided into four pieces in the same manner, placed on a surface opposite to the vibration plate 1211. Each of the upper PZT element 1212 and the lower PZT element 1213 is polarized in parallel to a direction directed to the vibration plate 1211 and causes a deformation by a piezoelectric effect with respect to an electric field in the direction.

A contact unit (distal end) 1215 that contacts the housing 16 is provided in one of short sides of the vibration plate 1211 in the substantially rectangular shape.

The ultrasonic vibrator 12 has holding units 1214 each of which is a projection formed in a node of standing wave vibration excited by the ultrasonic vibrator 12. Specifically, each of the holding units 1214 is provided at each center of two long sides of the vibration plate 1211. The holding unit 1214 is provided with a hole 1214a.

In the present embodiment, a size of the rectangular portion of the vibration plate 1211 of the ultrasonic vibrator 12 is 9 mm in the length and 2 mm in the width, and sizes of the upper and lower PZT elements are 8 mm in the length and 2 mm in the width. All of them has a thickness of 0.2 mm. The vibration plate 1211 is made from stainless steel and each of the PZT elements is made from a material that is generally referred to as hard-type lead zirconate titanate (Pb(Ti.Zr)O₃). However, such configurations are merely examples of configurations used for an experiment by the inventors of the invention and do not narrow the scope of the right of the invention. The invention is to be applied to an ultrasonic motor that rotates by receiving frictional force by preloading and the whole actuators.

### (Driving Principle)

The ultrasonic vibrator 12 has two types of vibration modes of a longitudinal-direction first vibration mode (hereinafter, referred to as expanding/contracting vibration) and a deflection (bending) third vibration mode (hereinafter, referred to as bending vibration). In the present embodiment, resonance frequencies of the expanding/contracting vibration and the bending vibration are coincident with each other as 240 kHz. Note that, such a numerical value is provided in the case of the aforementioned shapes and varies in accordance with design items, but does not affect propriety of application of the invention.

The vibration excited in the two types of vibration modes described above is standing wave vibration in which a position of the node does not vary. As described above, the holding unit 1214 is positioned at a location corresponding to the node of the standing wave vibration excited by the ultrasonic vibrator 12.

The expanding/contracting vibration is excited when the same voltage is applied to all the four-piece electrodes, and the bending vibration is excited when the same voltage is applied to electrodes located on each of diagonal lines of the four-piece electrodes and voltages having opposite polarities are applied to adjacent electrodes.

In the present embodiment, the electrodes located on each of the diagonal lines are short-circuited and the adjacent electrodes are isolated. Hereinafter, the voltages applied to the electrodes that are insulated are denoted by ϕ_{A} and ϕ_{B}. When alternating current of the same phase of 240 kHz is applied to ϕ_{A} and ϕ_{B}, the expanding/contracting vibration illustrated in FIG. 10 is excited, and in the case of reverse phase, the bending vibration illustrated in FIG. 11 is excited.

Thus, when the bending vibration is excited being shifting by ±90° relative to the expanding/contracting vibration, vibration in which the expanding/contracting vibration and the bending vibration are combined with the phase shifted by ±90° is excited. As a result, the contact unit 1215 of the ultrasonic vibrator is elliptically moved as illustrated in FIGS. 12(a) and (b).

Note that, though a method for driving the rectangular vibrator having four-piece electrodes is described here for convenience of the description, a driving method is not limited thereto as a main feature of the invention is to adopt the friction drive motor for driving in an internal contact manner. For example, ϕ_{A} and ϕ_{B} are set as sine waves, but are not limited thereto and may be square waves or sawtooth waves. Though the phase shift is set as ±90° for convenience of waveform generation, the phase shift is not limited thereto as conveyance is substantially enabled as long as the elliptical movement described above is caused. Further, there is also a method for enabling conveyance in two ways even in the case of a single phase, for example, by means of different vibration modes being established in accordance with a driving frequency. Derivatives thereof are also able to be applied to the invention.

### (Motor Cover)

The motor covers 1118 and 1119 are bases for supporting the pantograph preloading mechanisms 150 and 151 and have a function of protecting the ultrasonic vibrator 12 against contaminants such as blood.

Each of the motor covers 1118 and 1119 has a not-illustrated adjustment hole into which an adjustment screw 1514 for adjusting expanding/contracting of each of the pantograph preloading mechanisms 150 and 151 is inserted.

### (Housing 16)

The housing 16 serves as the rotor by itself and has a function of protecting the ultrasonic vibrator 12 against contaminants such as blood.

The housing 16 is desired to be made from a material having less wear because frictional force is received from the ultrasonic vibrator 12. According to examination by the inventors of the invention, it is effective to adopt, for example, steel subjected to high frequency hardening or dry carbon.

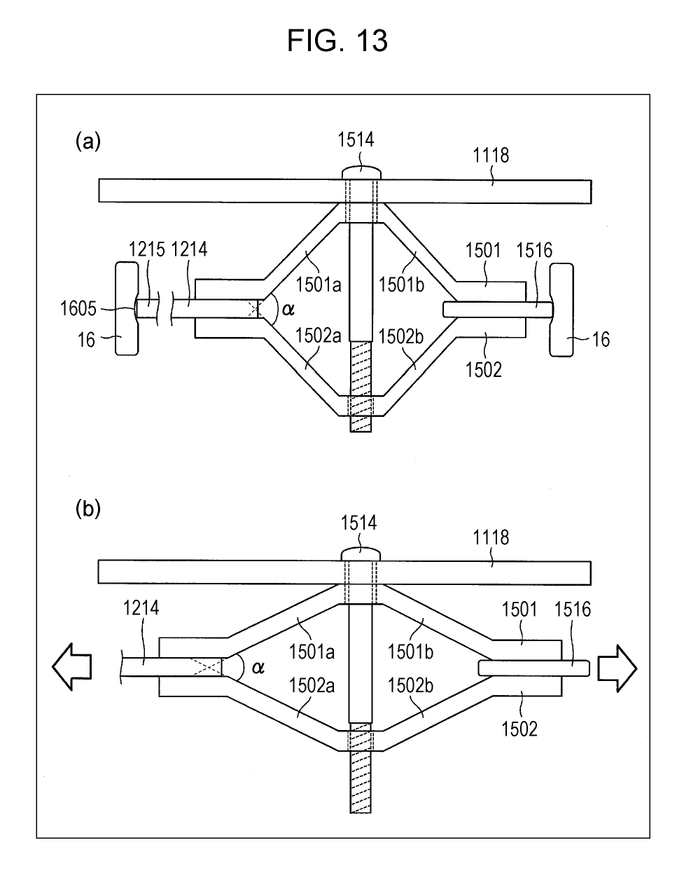

The housing 16 is provided with a guide groove 1605 (refer to FIG. 13) that restricts a position in contact with the contact unit 1215 of the ultrasonic vibrator 12. Thus, the ultrasonic vibrator 12 is able to rotate the housing 16 stably.

### (Pantograph Preloading Mechanisms 150 and 151)

FIGS. 13(a) and (b) are schematic views each illustrating an overview of the pantograph preloading mechanism 150. As illustrated in the figures, the pantograph preloading mechanism 150 includes metal fittings 1501 and 1502 each of which has a substantially V-shape, the adjustment screw (adjustment member) 1514, and a guide roller (slid support unit) 1516.

The metal fittings 1501 and 1502 respectively have arms 1501a and 1501b and arms 1502a and 1502b. The arm 1501a and the arm 1502a become paired and the arm 1501b and the arm 1502b also become paired. In this manner, each of the pantograph preloading mechanisms 150 and 151 includes two pairs of arms. One end of each of the pair of the arm 1501a and the arm 1502a is connected to the ultrasonic vibrator 12 with an angle formed, and the pantograph preloading mechanism 150 is configured to adjust pressure, with which the ultrasonic vibrator 12 is pressed against the housing 16, by adjusting an angle α formed between the arm 1501a and the arm 1502a at the end.

Each end of the arm 1501b and the arm 1502b is connected to the guide roller 1516 by a guide pin 1517 (refer to FIG. 8).

A configuration of the pantograph preloading mechanism 151 is similar to that of the pantograph preloading mechanism 150. Note that, the pantograph preloading mechanisms 150 and 151 may be single-arm pantographs including a pair of arms.

The present embodiment adopts a mechanism in which by tightening up the face-to-face metal fittings 1501 and 1502 by the adjustment screw 1514, a pantograph of the pantograph preloading mechanism 150 or 151 is expanded/contracted. As illustrated in FIG. 13, the adjustment screw 1514 is inserted into the adjustment hole formed in each of the motor covers 1118 and 1119 and a head of the adjustment screw 1514 (a part of the adjustment screw 1514) is exposed to each outer surface of the motor covers 1118 and 1119.

The pantograph preloading mechanisms 150 and 151 are connected to the holding units 1214 each formed in the node of vibration of the ultrasonic vibrator 12.

Specifically, the holding units 1214 are provided with the holes 1214a (refer to FIG. 9). The holes 1214a and holes (not illustrated) provided in one ends of the pantograph preloading mechanisms 150 and 151 are connected by guide pins 1518 (refer to FIG. 8). Each of the guide pins 1518 is a pin by which the ultrasonic vibrator 12 is connected to the holding unit 1214.

As described above, each of the holding units 1214 is provided at each center of the two long sides of the vibration plate 1211. That is, the holding units 1214 are formed to be positioned bilaterally symmetrical about a long axis of the ultrasonic vibrator 12 and the pantograph preloading mechanisms 150 and 151 are connected to each of the holding units 1214 that are paired.

Such configurations make it possible for the pantograph preloading mechanisms 150 and 151 to stably hold the ultrasonic vibrator 12.

As described above, the one end of the pantograph preloading mechanism 150 is connected to the hole of the holding unit 1214 of the ultrasonic vibrator 12 by the guide pin 1518. The other end of the pantograph preloading mechanism 150 is provided with the guide roller 1516 contacting an inner surface of the housing 16, by which the housing 16 is smoothly held.

In this manner, the pantograph preloading mechanisms 150 and 151 have one ends at which the holding units 1214 of the ultrasonic vibrator 12 are held and the other ends at which the housing 16 is held by the guide rollers. The housing 16 is held from an inner side at three portions in total of two portions of the guide rollers and the contact unit 1215 of the ultrasonic vibrator 12 and rotates.

At this time, as described above, the holding units 1214 are at positions corresponding to nodes of standing wave vibration excited by the ultrasonic vibrator 12. Thus, the pantograph preloading mechanisms 150 and 151 are able to hold the ultrasonic vibrator 12 without blocking vibration of the ultrasonic vibrator 12.

The metal fitting 1501 is bonded to the motor cover 1118, for example, by adhesive or a screw and a screw hole which are not illustrated. Each of the motor cover 1118 and the metal fitting 1501 is provided with a through hole and the metal fitting 1502 is provided with a screw hole, so that a threaded part of the adjustment screw 1514 is engaged with only the metal fitting 1502 and semi-fixed.

As indicated with a change from FIG. 13(a) to (b), by tightening the adjustment screw 1514, the pantograph preloading mechanism 150 is deformed so as to be laterally extended. That is, the pantograph preloading mechanism 150 is deformed so as to separate the ultrasonic vibrator 12 and the guide roller 1516 from each other. Actually, as a distance of the separation is almost fixed, when the adjustment screw 1514 is tightened up, the contact unit 1215 of the ultrasonic vibrator 12 is pressed against the housing 16 through elastic deformation of the metal fittings 1501 and 1502.

Specifically, each of the pantograph preloading mechanisms 150 and 151 generates force in a direction to widen a distance between the node (holding unit 1214) of the vibration of the ultrasonic vibrator 12 and the guide roller 1516 to thereby adjust pressure with which the ultrasonic vibrator 12 is pressed against the housing 16.

When the distance between the node of the vibration of the ultrasonic vibrator 12 and the guide roller 1516 is differentiated between the pantograph preloading mechanism 150 and the pantograph preloading mechanism 151, it is also possible to adjust a contact angle at which the contact unit 1215 is pressed against the housing 16.

Though shapes of the pantograph preloading mechanisms 150 and 151 are not limited as long as they do not depart from an object of preloading adjustment, it is desired that simplicity of manufacture and adjustment is considered.

### (Configuration of Controller Unit 130)

As illustrated in FIG. 1, the controller unit 130 includes an instruction input unit 131, a drive signal generation unit (voltage supply unit, operation instruction unit) 132, and a battery 133 which supplies power thereto. The controller unit 130 is detachably connected to the insertion unit conveyance unit 100 by a cable passing through the stand 102 and the flexible arm 101.

The instruction input unit 131 is an input device for inputting an instruction of an operator (user), and is, for example, an input device such as a joystick. For example, the operator manually tilts the joystick to back and forth or right and left, thereby inputting the instruction to convey (translate or rotate) the insertion unit 201 of the rigid endoscope 200. The instruction input unit 131 outputs the input instruction of the operator to the drive signal generation unit 132. For example, the input instruction of the operator designates a moving direction and a moving speed of the insertion unit 201.

On the basis of the input instruction of the operator, the drive signal generation unit 132 generates a drive signal for exciting desired vibration in the upper PZT element 1212 and the lower PZT element 1213, and applies the drive signal to the piezoelectric elements. The drive signal is an alternating voltage. The drive signal generation unit 132 decides a phase difference between two drive signals in accordance with the moving direction. The drive signal generation unit 132 decides amplitude of the voltage of the drive signal or a duty ratio of the drive signal in accordance with the moving speed.

As described above, the expanding/contracting vibration is caused when the same voltage is applied to all the four-piece electrodes, and the bending vibration is caused when the same voltage is applied to electrodes located on each of the diagonal lines of the four-piece electrodes and voltages having opposite polarities are applied to adjacent electrodes. When a direction of the elliptical movement of the contact unit 1215, which is caused by combination of the expanding/contracting vibration and the bending vibration, is changed in response to the input instruction of the operator, the rotation directions of the front in-wheel motor 1114 and the rear in-wheel motor 1115 are changed.

The drive signal generation unit 132 changes, on the basis of the instruction of the operator, drive signals supplied to the four-piece electrodes of each of the in-wheel motors, thereby changing the rotation direction of each of the in-wheel motors, and realizes the translation and rotation of the insertion unit 201 according to the instruction of the operator.

### (Effect of Differential Drive Mechanism 110A)

With the differential drive mechanism 110A according to the present embodiment, by appropriately setting shapes of the guides 1130 and 1131, appropriate crossing angles according to the outer diameter of the insertion unit 201 are automatically set. Thereby, an appropriate rotation speed according to the outer diameter of the insertion unit 201 is automatically set.

Moreover, with the differential drive mechanism 110A, any desired movement of the translation movement and the rotation movement of the insertion unit 201 is enabled to be selectively executed as the crossing angle by the front conveyance roller 1112 and the crossing angle by the rear conveyance roller 1113 are equal.

Further, with the differential drive mechanism 110A, driving by an appropriate combination of a rotation speed and a torque of a motor is able to be achieved whether the driving direction is the translation or the rotation. A rotation speed and a torque of a motor generally has an inverse correlation, and a DC motor or a stepping motor in which the inverse correlation between the rotation speed and the torque of the motor is linear is particularly desired to be driven by a combination of a rotation speed and a torque, with which the greatest force is provided or the highest power efficiency is achieved.

### Embodiment 2

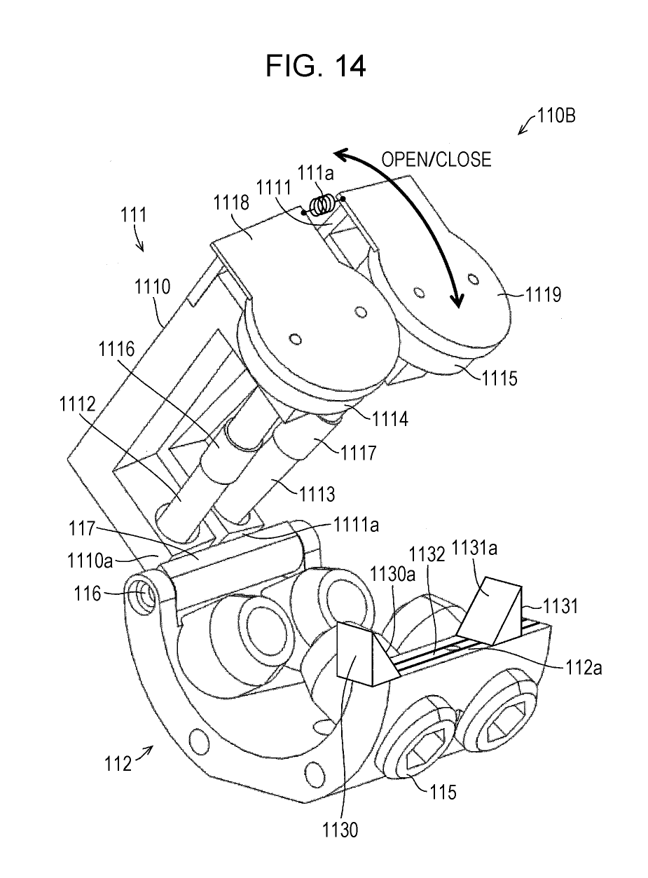

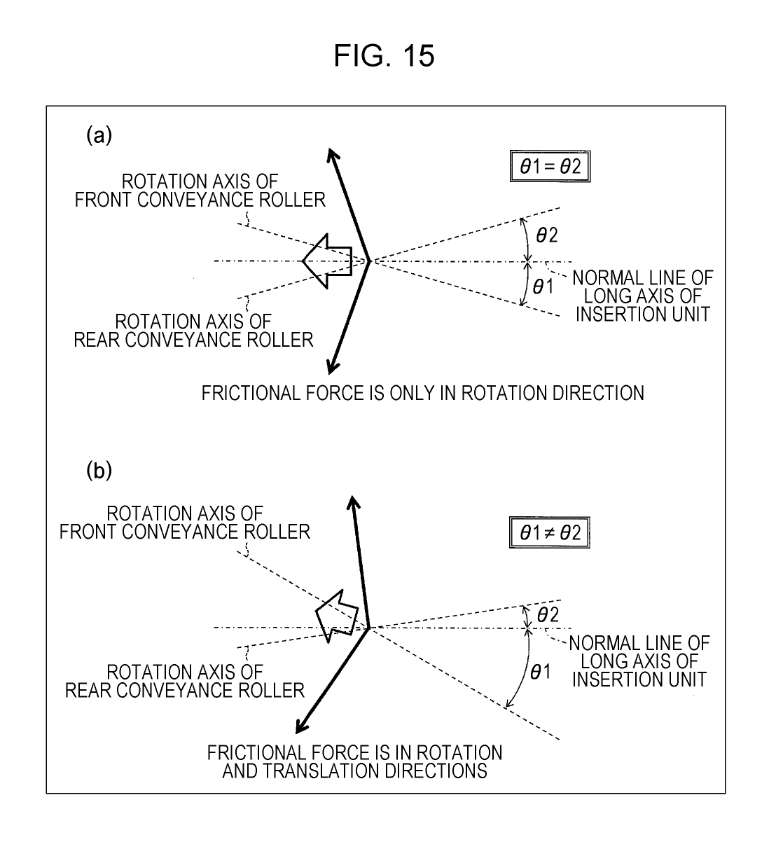

Another embodiment of the invention will be described below with reference to FIGS. 14 and 15. Note that, for convenience of description, members having the same functions as those of the members described in the aforementioned embodiment will be given the same reference signs and description thereof will be omitted.

A differential drive mechanism 110B according to the present embodiment includes a lane (guide unit) 1132 in addition to the configuration of the differential drive mechanism 110A described above. The guides 1130 and 1131 are configured to be movable on the lane 1132. With such a configuration, the front conveyance roller 1112 and the rear conveyance roller 1113 have different crossing angles θ.

### (Entire Configuration)

FIG. 14 illustrates an overview of the differential drive mechanism 110B according to the present embodiment.

As illustrated in FIG. 14, the differential drive mechanism 110B includes the upper housing unit 111, the lower housing unit 112, the coupling unit 117, and the preloading spring (restoring unit) 116.

The lower housing unit 112 includes four ball bearings (sliding bodies) 115, the guides 1130 and 1131, and the lane 1132. The lane 1132 is a groove for changing a distance between the guides 1130 and 1131. The lane 1132 is formed on the end surface 112a and is formed so as to be parallel to the long axis of the insertion unit 201 when the insertion unit 201 is inserted into the differential drive mechanism 110B.

The guides 1130 and 1131 and the lane 1132 constitute a main part of the aforementioned angle changing mechanism that changes the crossing angles. By moving the guides 1130 and 1131 along the lane 1132, the crossing angles are able to be changed so that the crossing angle of the front conveyance roller 1112 and the crossing angle of the rear conveyance roller 1113 are different from each other.

### (Operation of Angle Changing Mechanism)

FIGS. 15(a) and (b) each illustrates a resultant vector of frictional force when the front conveyance roller 1112 and the rear conveyance roller 1113 rotate as illustrated in FIG. 5. Here, FIG. 15(a) illustrates a case where a crossing angle θ1 of the front conveyance roller 1112 and a crossing angle θ2 of the rear conveyance roller 1113 are the same and FIG. 15(b) illustrates a case where the crossing angle θ1 and the crossing angle θ2 are different from each other.

Hereinafter, when the crossing angle θ1 and the crossing angle θ2 are the same, the crossing angles are considered to be symmetrical, and when the crossing angle θ1 and the crossing angle θ2 are different from each other, the crossing angles are considered to be unsymmetrical.

In a case where the crossing angles are symmetrical, when the front conveyance roller 1112 and the rear conveyance roller 1113 rotate in directions different from each other as illustrated in FIG. 5, the resultant vector of frictional force generated between the insertion unit 201 and each of the front conveyance roller 1112 and the rear conveyance roller 1113 is, as illustrated in FIG. 15(a), in a direction vertical to the long axis of the insertion unit 201, that is, a direction in which the insertion unit 201 rotates, so that the insertion unit 201 rotates about the long axis.

On the other hand, when the front conveyance roller 1112 and the rear conveyance roller 1113 rotate in the same direction with each other as illustrated in FIG. 4, the resultant vector is in a direction parallel to the long axis of the insertion unit 201, that is, a direction in which the insertion unit 201 is translated, so that the insertion unit 201 is translated in the direction parallel to the long axis.

That is, when the crossing angles are symmetrical, the insertion unit 201 performs only either the rotation movement or the translation movement in accordance with the rotation directions of the front conveyance roller 1112 and the rear conveyance roller 1113.

On the other hand, in the differential drive mechanism 110B according to the present embodiment, the crossing angles are able to be unsymmetrical by individually moving the guides 1130 and 1131 along the lane 1132 as described above.

When the front conveyance roller 1112 and the rear conveyance roller 1113 rotate in directions different from each other as illustrated in FIG. 5 in a state where the crossing angles are unsymmetrical, the resultant vector of frictional force generated between the insertion unit 201 and each of the front conveyance roller 1112 and the rear conveyance roller 1113 is in a direction oblique to both the long axis of the insertion unit 201 and the normal line thereof as illustrated in FIG. 15(b). That is, the resultant vector has both a vertical component and a horizontal component with respect to the long axis. In this case, the insertion unit 201 performs both the rotation and translation movement at the same time.

Thus, when the insertion unit 201 is inserted while being rotated at a fixed speed, it is only required that the positions of the guides 1130 and 1131 are decided in advance so that a desired ratio of the rotation speed and the translation speed is obtained. As a result, when inserting the insertion unit 201, the operator is able to easily perform the insertion operation of the insertion unit 201 without instructing or adjusting both the rotation and translation movement. An example of such an insertion unit 201 includes a thrombus removal catheter.

### Embodiment 3

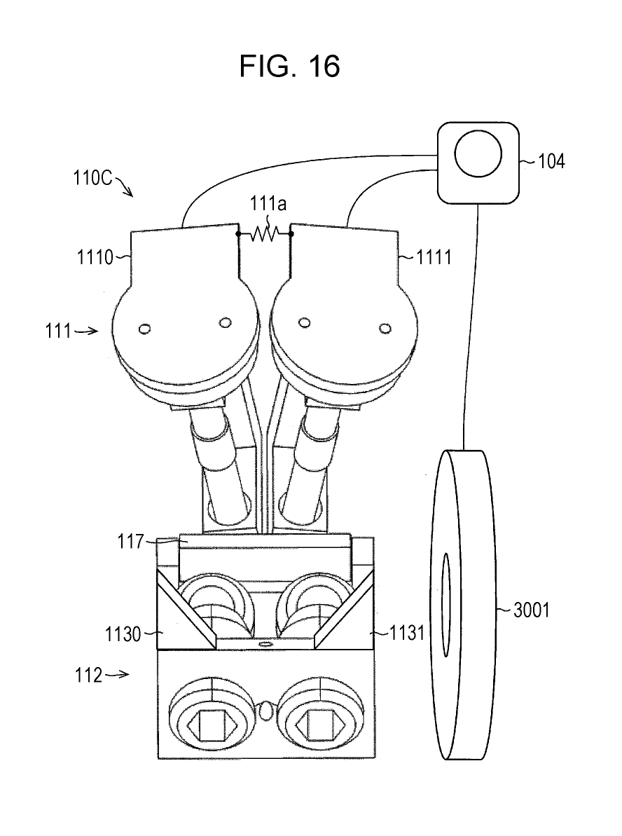

Another embodiment of the invention will be described below with reference to FIG. 16. Note that, for convenience of description, members having the same functions as those of the members described in the aforementioned embodiments will be given the same reference signs and description thereof will be omitted.

FIG. 16 illustrates a differential drive mechanism 110C according to the present embodiment. As illustrated in FIG. 16, the differential drive mechanism 110C according to the present embodiment includes an insertion unit movement detection sensor 3001 that detects a speed at which the insertion unit 201 is translated or rotates in addition to the configuration of the differential drive mechanism 110. With such a configuration, when there is a difference of a frictional coefficient between each of the conveyance rollers and the insertion unit 201 and the translation speed and the rotation speed of the insertion unit 201 are different from assumed speeds, the rotation speeds of the conveyance rollers are able to be corrected to suppress unintended movement of the insertion unit 201.

Specifically, the insertion unit movement detection sensor 3001 uses optical movement detection means whose technique has been established, for example, for an optical mouse for controlling a personal computer, or a non-contact measurement method such as a magnetic detection method.

When the optical movement detection means is used, the insertion unit movement detection sensor 3001 includes, for example, an image sensor and acquires an image on a surface of the insertion unit 201 at a sufficiently short and predetermined cycle. From matching regions in continuous images, the insertion unit movement detection sensor 3001 reads a movement amount of the insertion unit 201 between the images and calculates a movement speed of the insertion unit 201 on the basis of the movement amount and the cycle.

In general, on the assumption that frictional coefficients of two conveyance rollers are the same, rotation speeds of the conveyance rollers are set in a differential drive mechanism. However, it is considered that there is a difference between the frictional coefficients of the two conveyance rollers when contamination such as blood adheres to an insertion unit.

Thus, a controller unit 104 of the differential drive mechanism 110C according to the present embodiment monitors the translation speed and the rotation speed of the insertion unit 201 by the insertion unit movement detection sensor 3001, and, when scheduled movement is different from actual movement of the insertion unit 201, corrects the rotation speeds of the rollers. Accordingly, a safer treatment is able to be performed.

For example, according to arrangement in FIG. 5, in a case where unintended translation movement of the insertion unit 201 directed upward in FIG. 5 is detected when the insertion unit 201 is moved to rotate in a direction of an arrow, the speed at which the rear conveyance roller 1113 conveys the insertion unit 201 is considered to be higher than the speed at which the front conveyance roller 1112 conveys the insertion unit 201. Thus, by decreasing the rotation speed of the rear conveyance roller 1113 or increasing the rotation speed of the front conveyance roller 1112, the unintended translation movement is able to be suppressed.

### Embodiment 4

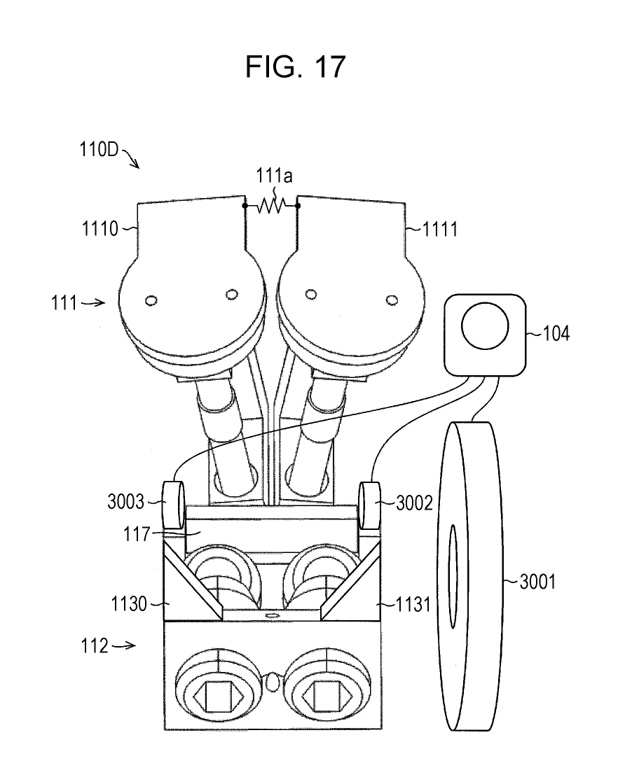

Another embodiment of the invention will be described below with reference to FIG. 17. Note that, for convenience of description, members having the same functions as those of the members described in the aforementioned embodiments will be given the same reference signs and description thereof will be omitted.

FIG. 17 illustrates a differential drive mechanism 110D according to the present embodiment. As illustrated in FIG. 17, the differential drive mechanism 110D according to the present embodiment includes pressing force adjustment mechanisms 3002 and 3003 that adjust force by which the front arm 1110 and the rear arm 1111 are pressed against the insertion unit 201, in addition to the configuration of the differential drive mechanism 110C. With such a configuration, when there is a difference of a frictional coefficient between each of the conveyance rollers and the insertion unit 201 and the translation speed and the rotation speed of the insertion unit 201 are different from assumed speeds, the pressing force of each of the conveyance rollers against the insertion unit 201 is able to be corrected to suppress the unintended movement of the insertion unit 201.

In general, on the assumption that frictional coefficients of two conveyance rollers are the same, rotation speeds of the conveyance rollers are set in a differential drive mechanism. However, it is considered that there is a difference between the frictional coefficients of the two conveyance rollers when contamination such as blood adheres to an insertion unit.

Thus, the controller unit 104 of the differential drive mechanism 110D according to the present embodiment monitors the translation speed and the rotation speed of the insertion unit 201 by the insertion unit movement detection sensor 3001, and, when scheduled movement is different from actual movement of the insertion unit 201, corrects the pressing force of the front arm 1110 and the rear arm 1111 by the pressing force adjustment mechanisms 3002 and 3003, thus making it possible to perform a safer treatment.

The pressing force adjustment mechanisms 3002 and 3003 are control mechanisms that control the pressing force of the front conveyance roller 1112 and the rear conveyance roller 1113 against the insertion unit 201. Specifically, each of the pressing force adjustment mechanisms 3002 and 3003 includes a spiral spring and a motor. The spiral spring has a center end connected to the motor and has a peripheral end connected to the front arm 1110 or the rear arm 1111.

When the motor rotates, in accordance with a direction of the rotation, force for closing or opening the front arm 1110 or the rear arm 1111 and the lower housing unit 112 is generated, so that force by which the front conveyance roller 1112 or the rear conveyance roller 1113 is pressed against the insertion unit 201 changes.

Thus, when the motor of the pressing force adjustment mechanism 3002 or 3003 rotates, force by which the front conveyance roller 1112 or the rear conveyance roller 1113 is pressed against the insertion unit 201 is able to be adjusted.

A signal which is output from the insertion unit movement detection sensor 3001 and related to the translation speed and the rotation speed of the insertion unit 201 is fed back to the controller unit 104, and the pressing force is corrected by the pressing force adjustment mechanisms 3002 and 3003 provided in the front arm 1110 and the rear arm 1111.

For example, according to the arrangement in FIG. 5, in a case where unintended translation movement of the insertion unit 201 directed upward in FIG. 5 is detected when the insertion unit 201 is moved to rotate in the direction of the arrow, the speed at which the rear conveyance roller 1113 conveys the insertion unit 201 is considered to be higher than the speed at which the front conveyance roller 1112 conveys the insertion unit 201. Thus, by decreasing the pressing force of the rear conveyance roller 1113 or increasing the pressing force of the front conveyance roller 1112, the unintended translation movement is able to be suppressed.

### CONCLUSION

An actuator (differential drive mechanism 110) according to an aspect 1 of the invention is an actuator including a plurality of conveyance rollers (front conveyance roller 1112, rear conveyance roller 1113) that are able to convey a rod-shaped operation element (insertion unit 201) in a long axis direction thereof and rotate the operation element about the long axis, and the actuator includes an angle changing mechanism that changes crossing angles of the plurality of conveyance rollers with respect to the operation element.

According to the aforementioned configuration, the actuator includes the plurality of conveyance rollers and the angle changing mechanism. The plurality of conveyance rollers are able to convey the operation element in the rod shape in the long axis direction thereof and rotate the operation element about the long axis. The angle changing mechanism changes the crossing angles of the plurality of conveyance rollers with respect to the operation element.

Thus, a ratio of a speed in a rectilinear-direction and a speed in a rotation-direction of the operation element is able to be easily adjusted.

The actuator according to an aspect 2 of the invention may further include a holding unit (ball bearing 115) that holds the operation element between each of the conveyance rollers and the holding unit, and a coupling unit (117) that changes distances from the respective conveyance rollers to the holding unit in accordance with a thickness of the operation element, in which the angle changing mechanism may change the crossing angles in conjunction with the distances, in the aspect 1.

According to the aforementioned configuration, the actuator further includes the holding unit and the coupling unit. The holding unit holds the operation element between each of the conveyance rollers and the holding unit. The coupling unit changes the distances from the respective conveyance rollers to the holding unit in accordance with the thickness of the operation element. The angle changing mechanism changes the crossing angles of the plurality of conveyance rollers with respect to the operation element in conjunction with the distances from the respective conveyance rollers to the holding unit.

Thus, the crossing angles of the conveyance rollers are able to be changed so that a component ratio of frictional force generated through rotation of the conveyance rollers between a translation direction and a rotation direction achieves an appropriate ratio according to the thickness of the operation element.

In the actuator according to an aspect 3 of the invention, the angle changing mechanism may include an urging unit (spring 111a) that urges the plurality of conveyance rollers so that a distance between distal ends of the conveyance rollers increases, and a restriction unit (guide 1130, 1131) that restricts the distance between the distal ends in accordance with the distances from the respective conveyance rollers to the holding unit, in the aspect 2.

According to the aforementioned configuration, the angle changing mechanism includes the urging unit and the restriction unit. The urging unit urges the plurality of conveyance rollers so that the distance between one distal ends of the conveyance rollers increases. The restriction unit restricts the distance between the distal ends in accordance with the distances from the respective conveyance rollers to the holding unit.

Thus, a simple configuration makes it possible to change the crossing angles of the conveyance rollers in accordance with the thickness of operation element.

In the actuator according to an aspect 4 of the invention, the restriction unit may include a pair of members that have inclined surfaces (1130a, 1131a) facing each other, in the aspect 3.

According to the aforementioned configuration, the restriction unit includes the pair of members. The pair of members have the inclined surfaces facing each other.

Thus, the crossing angles are able to be changed by guiding the conveyance rollers with the inclined surfaces.

The actuator according to an aspect 5 of the invention may further include a guide unit (lane 1132) that changes a distance between the pair of members, in the aspect 4.

According to the aforementioned configuration, the actuator includes the guide unit. The guide unit changes the distance between the pair of members as the restriction unit.

Thus, any relation between the thickness of the operation element and each of the crossing angles of the conveyance rollers is able to be set.

In the actuator according to an aspect 6 of the invention, the angle changing mechanism may change the crossing angles of the plurality of conveyance rollers so that the crossing angles are the same with each other, in any of the aspects 1 to 5.

According to the aforementioned configuration, the angle changing mechanism changes the crossing angles of the plurality of conveyance rollers of the actuator so that the crossing angles are the same with each other.

In this case, a component ratio of frictional force generated through rotation of the conveyance rollers between a translation direction and a rotation direction is the same in both the conveyance rollers.

Thus, when the plurality of conveyance rollers rotate at the same speed, either component of the frictional force of the translation direction or the rotation direction is canceled in accordance with the rotation direction of each of the conveyance rollers. As a result, it is possible to cause the operation element to perform either desired movement of the translation or the rotation.

In the actuator according to an aspect 7 of the invention, the angle changing mechanism may change the crossing angles of the plurality of conveyance rollers so that the crossing angles are different from each other, in any of the aspects 1 to 5.

According to the aforementioned configuration, the angle changing mechanism changes the crossing angles of the plurality of conveyance rollers of the actuator so that the crossing angles are different from each other.

In this case, a component ratio of frictional force generated through rotation of the conveyance rollers between the translation direction and the rotation direction varies with each of the conveyance rollers.

Thus, when the plurality of conveyance rollers rotate at the same speed, any components of the frictional force of the translation direction and the rotation direction are not cancelled to be zero, so that it is possible to cause the operation element to perform both the movement of the translation and the rotation at the same time.

The actuator according to an aspect 8 of the invention may further include a control mechanism (pressing force adjustment mechanism 3002, 3003) that controls pressing force of the conveyance rollers against the operation element, in any one of the aspects 1 to 7.

According to the aforementioned configuration, the actuator further includes the control mechanism. The control mechanism controls pressing force of the conveyance rollers of the actuator against the operation element.

Thus, when a frictional coefficient between each of the conveyance rollers and the operation element changes, for example, due to contamination of the operation element or the conveyance roller, the frictional force between each of the conveyance rollers and the operation element is able to be kept constant.

In the actuator according to an aspect 9 of the invention, a friction member (rubber roller 1116, 1117) having elasticity may be arranged on a surface of each of the conveyance rollers, in any of the aspects 1 to 8.

According to the aforementioned configuration, the friction member having elasticity is arranged on the surface of each of the conveyance rollers.

Thus, frictional force between the operation element and each of the conveyance rollers increases, so that idling of the conveyance rollers is able to be prevented.

In the actuator according to an aspect 10 of the invention, the friction member may be detachably arranged, in the aspect 9.

According to the aforementioned configuration, the friction member arranged on the surface of each of the conveyance rollers is detachable.

Thus, the friction member is able to be easily replaced in a case of damage or contamination.

In the actuator according to an aspect 11 of the invention, a step having a width equal to or greater than a width of the friction member may be provided on the surface of the conveyance roller, in the aspect 9 or 10.

According to the aforementioned configuration, the step is provided on the surface of the conveyance roller. The step has the width equal to or greater than the width of the friction member arranged on the surface of the conveyance roller.

Thus, a position of the friction member is able to be prevented from being significantly shifted due to friction with the operation element.

The actuator according to an aspect 12 of the invention may further include an ultrasonic motor (front in-wheel motor 1114, rear in-wheel motor 1115) that rotates the conveyance rollers, in any of the aspects 1 to 11.

According to the aforementioned configuration, the actuator includes the ultrasonic motor. The ultrasonic motor rotates the conveyance rollers.

Thus, no gear is required, so that a small-sized and silent device is realized.

The actuator according to an aspect 13 of the invention may include a first unit (upper housing unit 111) that has the plurality of conveyance rollers, and a second unit (lower housing unit 112) that has a holding unit holding the operation element between each of the conveyance rollers and the holding unit, in which the coupling unit may couple the first unit and the second unit so that relative positions of the first unit and the second unit are able to be changed, in the aspect 2.

According to the aforementioned configuration, the actuator includes the first unit and the second unit. The first unit has the plurality of conveyance rollers and the second unit has the holding unit holding the operation element between each of the conveyance rollers and the holding unit. The coupling unit couples the first and second units so that the relative positions of the first unit and the second unit are able to be changed.

Thus, when the operation element is held between each of the conveyance rollers of the first unit and the holding unit of the second unit, the relative positions of the first unit and the second unit are decided and distances from the respective conveyance rollers to the holding unit is decided.

The invention is not limited to the embodiments described above and may be modified in various manners within the scope of the claims, and an embodiment achieved by appropriately combining technical means disclosed in different embodiments is also encompassed in the technical scope of the invention. Further, by combining the technical means disclosed in each of the embodiments, a new technical feature may be formed.

### INDUSTRIAL APPLICABILITY

The invention is able to be used as a small-sized motor and is suitably used, in particular, in a medical device or a small-sized robot.

### REFERENCE SIGNS LIST

100 insertion unit conveyance unit
130 controller unit
110, 110A, 110B, 110C, 110D differential drive mechanism (actuator)
111 upper housing unit (first unit)
112 lower housing unit (second unit)
1112 front conveyance roller
1113 rear conveyance roller
1114 front in-wheel motor (ultrasonic motor)
1115 rear in-wheel motor (ultrasonic motor)
1116, 1117 rubber roller (friction member)
111a spring (urging unit)
115 ball bearing (holding unit)
12 ultrasonic vibrator
1130, 1131 guide (restriction unit)
1130a, 1131a inclined surface
1132 lane (guide unit)
117 coupling unit
150, 151 pantograph preloading mechanism
201 insertion unit (operation element)
3002, 3003 pressing force adjustment mechanism (control mechanism)

1. An actuator including a plurality of conveyance rollers that are able to convey a rod-shaped operation element in a long axis direction thereof and rotate the operation element about the long axis, the actuator comprising
an angle changing mechanism that changes crossing angles of the plurality of conveyance rollers with respect to the operation element, wherein the angle changing mechanism changes a resultant vector of the frictional force generated through rotation of the plurality of conveyance rollers by changing the crossing angles.

2. An actuator including a plurality of conveyance rollers that are able to convey a rod-shaped operation element in a long axis direction thereof and rotate the operation element about the long axis, the actuator comprising
an angle changing mechanism that changes crossing angles of the plurality of conveyance rollers with respect to the operation element, the actuator further comprising
a holding unit that holds the operation element between each of the conveyance rollers and the holding unit, and
a coupling unit that changes distances from the respective conveyance rollers to the holding unit in accordance with a thickness of the operation element, wherein
the angle changing mechanism changes the crossing angles in conjunction with the distances.

3. The actuator according to claim 2, wherein
the angle changing mechanism includes
an urging unit that urges the plurality of conveyance rollers so that a distance between distal ends of the conveyance rollers increases, and
a restriction unit that restricts the distance between the distal ends in accordance with the distances from the respective conveyance rollers to the holding unit.

4. The actuator according to claim 3, wherein the restriction unit includes a pair of members that have inclined surfaces facing each other.

5. The actuator according to claim 4, further comprising a guide unit that changes a distance between the pair of members.

6. The actuator according to claim 1, wherein the angle changing mechanism changes the crossing angles of the plurality of conveyance rollers so that the crossing angles are the same with each other.

7. An actuator including a plurality of conveyance rollers that are able to convey a rod-shaped operation element in a long axis direction thereof and rotate the operation element about the long axis, the actuator comprising
an angle changing mechanism that changes crossing angles of the plurality of conveyance rollers with respect to the operation element, wherein the angle changing mechanism changes the crossing angles of the plurality of conveyance rollers so that the crossing angles are different from each other.

8. The actuator according to claim 1, further comprising a control mechanism that controls pressing force of the conveyance rollers against the operation element.

9. The actuator according to claim 1, wherein a friction member having elasticity is arranged on a surface of the conveyance rollers.

10. The actuator according to claim 9, wherein the friction member is detachably arranged.

11. The actuator according to claim 9, wherein a step having a width equal to or greater than a width of the friction member is provided on the surface of the conveyance roller.

12. The actuator according to claim 1, further comprising an ultrasonic motor that rotates the conveyance rollers.

13. The actuator according to claim 2, comprising
a first unit that has the plurality of conveyance rollers, and
a second unit that has a holding unit holding the operation element between each of the conveyance rollers and the holding unit, wherein
the coupling unit couples the first unit and the second unit so that relative positions of the first unit and the second unit are able to be changed.
